(19) United States
(12) Patent Application Publication
(10) Pub. No.: US 2021/0290825 A1
(43) Pub. Date: Sep. 23, 2021

(54) INJECTION FORMULATION COMPOSITION FOR USE AS FILLER OR DRUG CARRIER THROUGH CLICK CHEMISTRY REACTION

(71) Applicant: AJOU UNIVERSITY INDUSTRY-ACADEMIC COOPERATION FOUNDATION, Gyeonggi-do (KR)

(72) Inventors: Moon Suk KIM, Suwon-si (KR); Seung Hun Park, Seoul (KR); Hyeon Jin JU, Changwon-si (KR)

(73) Assignee: AJOU UNIVERSITY INDUSTRY-ACADEMIC COOPERATION FOUNDATION, Gyeonggi-do (KR)

(21) Appl. No.: 17/053,429

(22) PCT Filed: Jun. 25, 2019
(86) PCT No.: PCT/KR2019/007631

§ 371 Date: Nov. 6, 2020

(30) Foreign Application Priority Data
May 8, 2018 (KR) 10-2018-0052631
Feb. 27, 2019 (KR) 10-2019-0023035

(51) Int. Cl. A61L 27/52; A61L 27/20; A61L 27/56; A61L 27/54

(52) U.S. Cl. CPC A61L 27/52; A61L 27/20; A61L 2400/06; A61L 27/54; A61L 27/56

(57) ABSTRACT

The present invention relates to an injection formulation composition for use as a filler or a drug carrier through a click chemistry reaction. More specifically, the present invention relates to an injection formulation composition comprising: a first liquid comprising a first biopolymer having a first click chemistry functional group introduced thereinto; and a second liquid comprising a second biopolymer having a second click chemistry functional group introduced thereinto, wherein the first click chemistry functional group is chemically linkable with the second click chemistry functional group, to a method for preparing an injection formulation hydrogel using the composition, and to a medical filler, an in-vivo injection type supporter, or a drug carrier using the composition.

### TECHNICAL FIELD

The present invention relates to an injectable composition for use as a filler or a drug carrier through a click chemistry reaction, and more particularly to: an injectable composition including: a first liquid including a first biopolymer into which a first click chemistry functional group is introduced; and a second liquid including a second biopolymer into which a second click chemistry functional group is introduced, wherein the first click chemistry functional group and the second click chemistry functional group are chemically linkable; a method of preparing an injectable hydrogel using the same; and a medical filler, bio-injectable scaffold or drug carrier using the same.

### BACKGROUND ART

Recently, hyaluronic acid injectable hydrogels are receiving a lot of interest in the medical field, and are expected to be widely used in fields ranging from medical fillers to drug (including a biotransmitter) carriers, and organ/tissue regeneration using a three-dimensional structure. Such an injectable hydrogel has the advantage that it can be simply injected into a living body using a syringe or the like without a surgical procedure.

In general, in the case of an injectable hydrogel, it can be implanted using a syringe because it has fluid-like properties in vitro, and gelation occurs after injection into the body. That is, after implantation, the hydrogel can serve as a drug carrier for sustained release of a drug (bioactive substance) or a scaffold capable of maintaining cell growth. In addition, by employing various crosslinking methods, physicochemical properties such as gelation time, a swelling ratio, decomposition, and mechanical properties of the hydrogel can be controlled, and the possibility of controlling these physicochemical properties provides a great advantage in the use of a hydrogel in drug delivery systems or tissue engineering according to applications. Therefore, it is important to prepare a hydrogel having desired physicochemical properties by controlling an appropriate degree of crosslinking.

Injectable hydrogels may be obtained through reversible interactions by non-covalent bonding (hydrophobic bonding, hydrogen bonding, or the like) or irreversible bonding by a chemical reaction (heat, UV). Among these, an injectable hydrogel prepared through the reversible bonding uses toxic additives such as a photo-initiator and a cross-linker, and has a problem in that mechanical properties are weak.

Therefore, there is a need for a crosslinked hydrogel capable of easily controlling the physicochemical properties of a biocompatible material while overcoming the above-described problems.

### DESCRIPTION OF EMBODIMENTS

### Technical Problem

The present invention provides an injectable composition including: a first liquid including a first biopolymer into which a first click chemistry functional group is introduced; and a second liquid including a second biopolymer into which a second click chemistry functional group is introduced, wherein the first click chemistry functional group and the second click chemistry functional group are chemically linkable.

However, technical problems to be solved by the present invention are not limited to the above-described technical problems, and other unmentioned technical problems will become apparent to those of ordinary skill in the art from the following description.

### Technical Solution

According to an aspect of the present disclosure, there is provided an injectable composition including: a first liquid including a first biopolymer into which a first click chemistry functional group is introduced; and a second liquid including a second biopolymer into which a second click chemistry functional group is introduced, wherein the first click chemistry functional group and the second click chemistry functional group are chemically linkable.

The present invention also provides a method of preparing an injectable hydrogel, including: (a) preparing a first liquid by adding a material including a first click chemistry functional group to a first biopolymer; (b) preparing a second liquid by adding a material including a second click chemistry functional group to a second biopolymer; and (c) reacting the first liquid and the second liquid to chemically link the first click chemistry functional group and the second click chemistry functional group.

The present invention also provides a medical filler or bio-injectable scaffold including the injectable hydrogel.

The present invention also provides a drug carrier using the injectable hydrogel and a drug.

### Advantageous Effects of Invention

An injectable hydrogel according to the present invention is simply prepared from an injectable composition through a click chemistry reaction, and by adjusting the type and number of click chemistry functional groups, the molecular weight of a biopolymer, and the like, the physicochemical properties of the injectable hydrogel can be easily controlled. Therefore, the injectable hydrogel according to the present invention is suitable as a medical filler or a bio-injectable scaffold, and may further include a drug (including a biotransmitter), and thus can also be used as a sustained-release drug carrier or the like.

### BRIEF DESCRIPTION OF DRAWINGS

FIG. 1 is a view illustrating a mechanism of action of a specific crosslinked hyaluronic acid hydrogel through a click chemistry reaction of hyaluronic acid into which a first click chemistry functional group is introduced and hyaluronic acid into which a second click chemistry functional group is introduced according to the present invention.

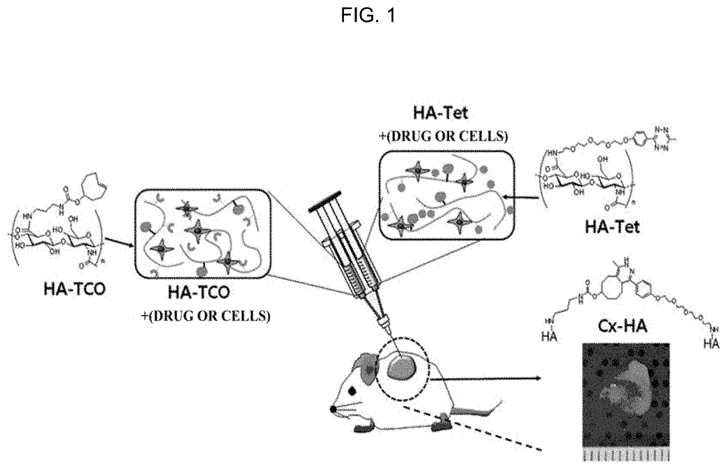

FIG. 2 illustrates ¹H-NMR results of a specific crosslinked hyaluronic acid hydrogel prepared according to an embodiment of the present invention.

FIG. 3 is a graph showing the rheological properties of a specific crosslinked hyaluronic acid hydrogel prepared according to an embodiment of the present invention.

FIG. 4 illustrates images of a specific crosslinked hyaluronic acid hydrogel prepared according to an embodiment of the present invention, observed using a scanning electron microscope.

FIG. 5 is a graph showing water swelling property of a specific crosslinked hyaluronic acid hydrogel prepared according to an embodiment of the present invention with respect to water.

FIG. 6 illustrates image showing the in-vivo gel shape retention characteristics of a specific crosslinked hyaluronic acid hydrogel prepared according to an embodiment of the present invention.

FIG. 7 illustrates images showing the in-vivo retention properties of a specific crosslinked hyaluronic acid hydrogel prepared according to an embodiment of the present invention.

FIG. 8 is a graph showing the cumulative release characteristics of an anticancer agent (doxorubicin) from a specific crosslinked hyaluronic acid hydrogel containing the anticancer agent (doxorubicin) prepared according to an embodiment of the present invention.

FIG. 9 is a graph showing the inhibition of growth of cancer cells by a specific crosslinked hyaluronic acid hydrogel containing an anticancer agent (doxorubicin) prepared according to an embodiment of the present invention.

FIG. 10 is a graph showing the inhibition of growth of cancer formed in animals by injection of a specific crosslinked hyaluronic acid hydrogel containing an anticancer agent (doxorubicin) prepared according to an embodiment of the present invention.

FIG. 11 is a graph showing the results of comparing the inhibition of growth of cancer formed in animals by injection of a specific crosslinked hyaluronic acid hydrogel containing an anticancer agent (doxorubicin) prepared according to an embodiment of the present invention with the inhibition of growth of cancer formed in animals by injection of a composition containing an anticancer agent (doxorubicin) in a freeze-dried pulverized product of a specific crosslinked hyaluronic acid hydrogel prepared according to an embodiment of the present invention.

FIG. 12 is a set of images showing the results of observing that the fluorescence of an anticancer agent (doxorubicin) appeared in respective organs of animals after injection of a specific crosslinked hyaluronic acid hydrogel containing an anticancer agent (doxorubicin) prepared according to an embodiment of the present invention.

FIG. 13 is a set of graphs showing the HPLC results of quantitatively confirming the amount of an anticancer agent (doxorubicin) observed in respective organs of animals after injection of a specific crosslinked hyaluronic acid hydrogel containing the anticancer agent (doxorubicin) prepared according to an embodiment of the present invention.

FIG. 14 is a set of graphs showing the results of quantitatively confirming changes in the blood glucose level of diabetic-induced animals after injection of an insulin-containing specific crosslinked hyaluronic acid hydrogel prepared according to an embodiment of the present invention.

### BEST MODE

While having continued research on fillers (medical fillers or bio-injectable scaffolds) and drug carriers, the inventors of the present invention confirmed that, by introducing different click chemistry functional groups to biopolymers, and chemically crosslinking the functional groups through a click chemistry reaction, a hydrogel that is easy to prepare and has excellent bio-sustainability can be prepared, and thus completed the present invention.

Hereinafter, the present invention will be described in detail.

The present invention provides an injectable composition including: a first liquid including a first biopolymer into which a first click chemistry functional group is introduced; and a second liquid including a second biopolymer into which a second click chemistry functional group is introduced, wherein the first click chemistry functional group and the second click chemistry functional group are chemically linkable.

The injectable composition is a composition for simply preparing an injectable hydrogel, and the first liquid and the second liquid may be present in a state separated from each other in a dual syringe, and then may be mixed. When mixed, the first click chemistry functional group and the second click chemistry functional group in the injectable composition may form a specific crosslinking bond through a click chemistry reaction, thereby being prepared in the form of an injectable hydrogel. The hydrogel is a gel using water as a dispersion medium, and the gel is a state in which a sol loses fluidity and is solidified, and the solubility of a dispersed phase is lowered, thereby forming a network structure via linkages and the dispersion medium is contained therein.

The first click chemistry functional group includes one or more selected from an alkyne group, an epoxy group, an acryloyl group, and a tetrazine group, and the second click chemistry functional group may include one or more selected from an azide group, a thiol group, an amine group, and a cyclooctene group.

Specifically, as the first click chemistry functional group, the alkyne group may be derived from a material containing a first click chemistry functional group selected from amino-PEG4-alkyne, alkyne-PEG5-acid, and alkyne-PEG-amine; the epoxy group may be derived from a material containing a first click chemistry functional group selected from oxiranylamine and 2-oxiranyl-ethylamine; the acryloyl group may be derived from a material containing a first click chemistry functional group selected from acrylamide, acrylic acid, and acryloyl chloride; and the tetrazine group may be derived from a material containing a first click chemistry functional group selected from methyltetrazine-amine, meathyltetrazine-PEG4-amine, methyltetrazinepropylamine, tetrazine-PEG5-NHS ester, methyltetrazine-PEG4-NHS ester, methyltetrazine-silfo-NHS ester, methlytetrazine-PEG4-acid, methyltetrazine-PEG12-NHS ester, methyltetrazine-NHS ester, methyltetrazine-acid, and tetrazine-acid.

In one embodiment of the present invention, as the material containing a first click chemistry functional group, methyltetrazine-PEG4-amine (Tet) was used.

In addition, as the second click chemistry functional group, the azide group may be derived from a material containing a second click chemistry functional group selected from azide-PEG4-amine; the thiol group may be derived from a material containing a second click chemistry functional group selected from 3-amino-1-propanethiol, 11-mercaptoundecanoic acid, amino-methanethiol, and thiol PEG amine; the amine group may be derived from a material containing a second click chemistry functional group selected from ethylene diamine, PEG diamine, (S)-3-amino-2-(hydroxymethyl)propionic acid and amino-acetic acid; and the cyclooctene group may be derived from a material containing a second click chemistry functional group selected from trans-cyclooctene-amine, trans-cyclooctene-NHSester, trans cyclooctene-PEG-NHS ester, and trans cyclooctene-PEG4-acid.

In one embodiment of the present invention, as the material containing the second click chemistry functional group, trans-cyclooctene-amine (TCO) was used.

The first click chemistry functional group and the second click chemistry functional group are able to be chemically crosslinked with each other, and such chemical bonding may be made within a short time, particularly within a few seconds, and it is advantageous in that the first liquid and the second liquid can be gelled within a short time. Specifically, a combination of the first click chemistry functional group and the second click chemistry functional group may be one or more selected from: an alkyne group and an azide group; an alkyne group and a thiol group; an epoxy group and an amine group; an epoxy group and a thiol group; an acryloyl group and an amine group; an acryloyl group and a thiol group; and tetrazine and cyclooctene.

In one embodiment of the present invention, as the combination of the first click chemistry functional group and the second click chemistry functional group, tetrazine and cyclooctene were used, and the groups were reacted with each other to form specific cross-linked bonds.

The first biopolymer and the second biopolymer may be the same or different from each other, and it is preferable that the first biopolymer and the second biopolymer each independently include one or more selected from hyaluronic acid, small intestine submucosal tissue, carboxymethylcellulose, alginate, chitosan, polyacrylamide, poly(N-isopropylacrylamide), and β-glycerophosphate, but the present invention is not limited thereto.

More specifically, the first biopolymer and the second biopolymer may have the same molecular weight or different molecular weights before the introduction of the functional groups, and the molecular weight thereof is preferably in the range of 500,000 Da to 6,000,000 Da, but the present invention is not limited thereto. When the molecular weight of the biopolymer is less than the above range, there is a problem that physical properties are too low, and when the molecular weight exceeds the above range, there is a problem in use due to an increase in viscosity.

By adjusting the number of first click chemistry functional groups introduced with respect to 1 mole of the first biopolymer or the number of second click chemistry functional groups introduced with respect to 1 mole of the second biopolymer, the degree of crosslinking of the biopolymer hydrogel may be controlled, and consequently, physical properties may be optimized according to the use of the hydrogel. Specifically, the number of first click chemistry functional groups introduced with respect to 1 mole of the first biopolymer or the number of second click chemistry functional groups introduced with respect to 1 mole of the second biopolymer may be in the range of 100 to 2,000, and most preferably in the range of 100 to 300 in terms of easy injection via a syringe, but the present invention is not limited thereto.

Specifically, when the biopolymer hydrogel has a low degree of click crosslinking, the biopolymer hydrogel is applicable to various soft tissues due to low strength and a high swelling ratio. Meanwhile, when the biopolymer hydrogel has an intermediate degree of click crosslinking, the biopolymer hydrogel may be applied to cartilage tissue and used for the prevention or treatment of arthritis, cartilage damage, or cartilage defects. On the other hand, when the biopolymer hydrogel has a high degree of click crosslinking, the biopolymer hydrogel may be applied to bone tissue due to high strength and a low swelling ratio, and used for the prevention or treatment of osteoporosis or bone defect disease.

A drug may be additionally included in the first liquid and the second liquid, and in this case, the drug refers to a broad concept including not only general drugs but also biotransmitters. Known drugs may be used as the drug, and for example, the drug may include a chemical drug, a protein drug, a peptide drug, a nucleic acid molecule for gene therapy, and nanoparticles. For example, the drug includes, but is not limited to, an anticancer agent, a therapeutic agent for diabetic diseases, an anti-inflammatory agent, an analgesic agent, an antiarthritic agent, an antispasmodic agent, an antidepressant, an antipsychotic drug, a tranquilizer, an antianxiety drug, a narcotic antagonist, anti-Parkinson's disease drug, a cholinergic agonist, an anti-angiogenesis inhibitor, an immunosuppressant, an antiviral agent, an antibiotic, an appetite suppressant, an anticholinergic drug, an antihistamine agent, an anti-migraine agent, a hormonal drug, a coronary, cerebrovascular or peripheral vasodilator, a contraceptive, an antithrombotic drug, a diuretic drug, an antihypertensive drug, a cardioprotective agent, and cosmetic ingredients (e.g., an anti-wrinkle agent, an anti-skin-aging agent, and a skin whitening agent).

Specifically, the drug preferably includes: an anticancer agent including one or more selected from doxorubicin, cisplatin, paclitaxel, vincristine, topotecan, docetaxel, 5-fluorouracil (5-FU), Gleevec, carboplatin, daunorubicin, valrubicin, flutamide, and gemcitabine; or a therapeutic agent for diabetic diseases including insulin or an insulinotropic peptide, but the present invention is not limited thereto.

In one embodiment of the present invention, doxorubicin and insulin were used as the drugs.

The present invention also provides a method of preparing an injectable hydrogel, including: (a) preparing a first liquid by adding a material containing a first click chemistry functional group to a first biopolymer; (b) preparing a second liquid by adding a material containing a second click chemistry functional group to a second biopolymer; and (c) reacting the first liquid and the second liquid to chemically link the first click chemistry functional group and the second click chemistry functional group.

In process (a), the material containing the first click chemistry functional group may be one or more selected from amino-PEG4-alkyne, alkyne-PEG5-acid, alkyne-PEG-amine, oxiranylamine, 2-oxiranyl-ethylamine, acrylamide, acrylic acid, acryloyl chloride, methyltetrazine-amine, methyltetrazine-PEG4-amine, methyltetrazine-propylamine, tetrazine-PEG5-NHS ester, methyltetrazine-PEG4-NHS ester, methyltetrazine-silfo-NHS ester, methyltetrazine-PEG4-acid, methyltetrazine-PEG12-NHS ester, methyltetrazine-NHS ester, methyltetrazine-acid, and tetrazine-acid.

In process (b), the material containing the second click chemistry functional group may be one or more selected from azide-PEG4-amine, 3-amino-1-propanethiol, 11-mercaptoundecanoic acid, amino-methanethiol, thiol PEG amine, ethylene diamine, PEG diamine, (S)-3-amino-2-(hydroxymethyl)propionic acid, amino-acetic acid, transcyclooctene-amine, trans-cyclooctene-NHS ester, trans cyclooctene-PEG-NHS ester, and trans cyclooctene-PEG4-acid.

Optionally, in process (a) or process (b), a condensing agent or a drug may be additionally added. Specifically, in order to introduce a first or second click chemistry functional group into the first or second biopolymer, it is preferable to perform a condensation reaction by additionally including a condensing agent in the first or second biopolymer solution, but the present invention is not limited thereto. As the condensing agent, 4-(4,6-dimethoxy-1,3,5-triazin-2-yl)-4-methylmorpholinium chloride (DMTMM) may be used, and the amount of condensing agent may be adjusted to control the degree of a condensation reaction. Meanwhile, the drug is the same as described above, and can be applied with a drug carrier by injection of the drug.

The injectable hydrogel prepared using the method is not only in the form of a gel, but is also an injectable formulation.

Specifically, the injectable hydrogel has a high storage modulus (G′), and the storage modulus (G′) of the injectable hydrogel in the frequency range of 0.1 Hz to 10 Hz, measured using a rheometer, may range from 2.00×10² Pa to 2.00×10³ Pa, and preferably ranges from 2.00×10² Pa to 4.00×10² Pa, but the present invention is not limited thereto.

In addition, the complex viscosity of the injectable hydrogel at 25° C. may range from 3.00×10¹ Pa·s to 3.0×10² Pa·s, and preferably is in the range of 3.00×10¹ Pa·s to 6.0×10¹ Pa·s, but the present invention is not limited thereto.

In addition, the injectable hydrogel may be in a porous form. This means that crosslinking has occurred.

In addition, a swelling ratio of the injectable hydrogel with respect to water may range from 2,000% to 10,000%, preferably 7,500% to 10,000%, but the present invention is not limited thereto.

The present invention also provides a medical filler or bio-injectable scaffold (filler) including the prepared injectable hydrogel.

The present invention also provides a drug carrier including the prepared injectable hydrogel and a drug.

The drug may be present inside the hydrogel, or may be present in a form mixed with the hydrogel, but being present inside the hydrogel is preferable in terms of effective drug delivery, but the present invention is not limited thereto.

The drug is the same as described above.

The drug carrier may be used as a sustained-release drug carrier, and may have a cumulative drug release rate of 50% to 100% for 28 days. Specifically, the drug carrier may have a cumulative drug release rate of 50% to 80% for 16 days, and may have a cumulative drug release rate of 80% to 100% for 28 days, and thus the drug is slowly released over a long period of time so that efficacy is maintained for a long period of time. Thus, it is advantageous in that the occurrence of side effects by single/repeated injection of the drug can be completely prevented.

Furthermore, the present invention provides a use of the injectable hydrogel or the drug carrier for the treatment of cancer, diabetes, or diseases.

The present invention also provides a method of treating cancer or a diabetic disease, including administering, to an individual, the injectable hydrogel or the drug carrier.

In the present invention, “individual” means a subject in need of treatment for a disease, and more particularly, includes mammals such as humans, non-human primates, mice, rats, dogs, cats, horses, cows, and the like.

Hereinafter, the present invention will be described in more detail with reference to the following examples. These examples are provided for illustrative purposes only, and it will be obvious to those of ordinary skill in the art that the scope of the present invention is not construed as being limited by these examples.

### Examples 1 to 3. Preparation of Specific Crosslinked Hyaluronic Acid Hydrogels Having First and Second Click Chemistry Functional Groups Introduced Thereinto

(1) 100 mg of hyaluronic acid (HA) (molecular weight: 1,000,000 Da) was added to 10 mL of tertiary distilled water, and then stirred at 25° C. for 24 hours to prepare a hyaluronic acid solution, which was then distributed into vials in an amount of 5 mL/vial.

(2) 8.3 mg of 4-(4,6-dimethoxy-1,3,5-triazin-2-yl)-4-methylmorpholinium chloride (DMTMM) as a condensing agent and 9.93 mg of methyltetrazine-PEG4-amine (Tet) were added to the vials with the hyaluronic acid solution contained therein, and stirred at 25° C. for 24 hours to allow a reaction to occur. Next, the reaction product was dialyzed for 72 hours, and then lyophilized at −80° C., thereby completing the preparation of HA-Tet into which a first click chemistry functional group was introduced.

As shown in Table 1 below, the amount of the click functional group introduced into HA-Tet having the first click chemistry functional group introduced thereinto was analyzed through elemental analysis of a Flash EA1112 device (CE Instruments, Italy).

(3) Hyaluronic acid into which a chemical functional group was introduced was prepared using the same method as described above, except that 7.7 mg of trans-cyclooctene-amine (TCO) was added instead of Tet, thereby completing the preparation of HA-TCO into which a second click chemistry functional group was introduced.

As shown in Table 1 below, the amount of the click functional group introduced into HA-TCO having the second click chemistry functional group introduced thereinto was analyzed through elemental analysis of a Flash EA1112 device (CE Instruments, Italy).

(4) The prepared HA-Tet having the first click chemistry functional group introduced thereinto and the prepared HA-TCO having the second click chemistry functional group introduced thereinto were each dissolved in 2% physiological saline, and then were placed in a dual syringe to allow HA-Tet and HA-TCO to be mixed, thereby inducing a click chemistry reaction, and a hyaluronic acid hydrogel of the present invention was finally prepared through specific crosslinking.

(5) The degree of crosslinking of the hyaluronic acid hydrogel prepared as above is adjusted according to the number of first or second click chemistry functional groups (Tet or TCO) introduced.

TABLE 1

| | | | | | C/N | |
|---|---|---|---|---|---|---|
| | | | | | mole | Injection |
| | Sample | C (%) | H (%) | N (%) | ratio | NO |
| Comparative | HA | 34.7 | 5.2 | 2.9 | 14.0 | |
| Example 1 | | | | | | |
| Example 1 | HA-Tet₂₅₀ | 39.5 | 5.5 | 4.4 | 10.5 | Cx-HA-250 |
| | HA-TCO₂₅₀ | 42.6 | 5.8 | 4.0 | 12.4 | |
| Example 2 | HA-Tet₅₀₀ | 39.4 | 5.9 | 4.5 | 10.2 | Cx-HA-500 |
| | HA-TCO₅₀₀ | 39.5 | 5.7 | 3.9 | 11.8 | |
| Example 3 | HA-Tet₁₀₀₀ | 40.6 | 5.5 | 5.5 | 8.5 | Cx-HA-1000 |
| | HA-TCO₁₀₀₀ | 40.6 | 6.2 | 4.4 | 10.7 | |

* Number: number of first or second click chemistry functional groups (Tet or TCO)
introduced per mole of hyaluronic acid (HA) (250, 500, 1000)

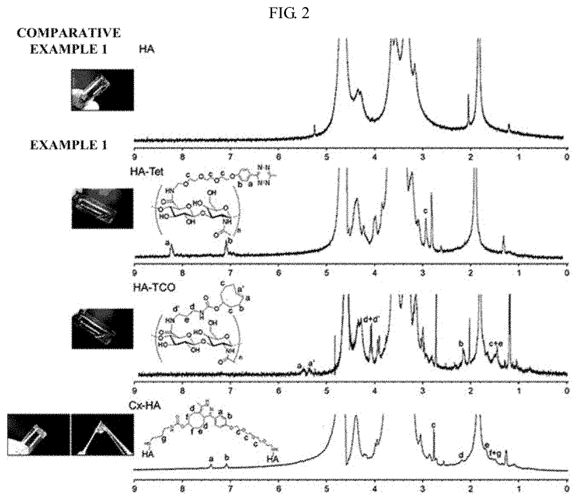

In addition, through ¹H-NMR analysis, it was confirmed that HA-Tet had the first click chemistry functional group introduced thereinto and HA-TCO had the second click chemistry functional group introduced thereinto (FIG. 2).

### Example 4. Preparation of Specific Crosslinked Hyaluronic Acid Hydrogel with Fluorescent Material Introduced for Near-Infrared Fluorescence Imaging

In a 50 mL round-bottom flask, IR783 (80 mg, 0.11 mmol) and 4-mercaptobenzoic acid (51 mg, 0.33 mmol) were dissolved in DMF (3 mL) and stirred at room temperature for 24 hours. In order to remove unreacted substances and impurities from the reacted solution, the resulting solution was precipitated in 30 mL of a mixed solvent of ethanol and diethyl ether (v/v=1/19) and filtered. The green solid obtained as the filtrate was vacuum-dried to prepare IR783-COOH.

Next, HA-Tet with the first click chemistry functional group introduced thereinto and HA-TCO with the second click chemistry functional group introduced thereinto, which were prepared according to Example 1, were each added to 10 mL of tertiary distilled water to prepare a solution, and 3.3 mg of 4-(4,6-dimethoxy-1,3,5-triazin-2-yl)-4-methylmorpholinium chloride (DMTMM) was added as a condensing agent to each vial. While each solution was slowly added dropwise to 5 mL of tertiary distilled water containing 10.5 mg of IR783-COOH, the resultant solution was stirred at 25° C. for 24 hours to allow a reaction to occur, followed by dialysis for 3 days and lyophilization at −80° C., thereby completing the preparation of NIR-HA-Tet having the first click chemistry functional group and IR783 introduced thereinto and NIR-HA-TCO having the second click chemistry functional group and IR783 introduced thereinto.

### Example 5. Preparation of Specific Crosslinked Hyaluronic Acid Hydrogel Containing Anticancer Agent

HA-Tet (2 mg) with the first click chemistry functional group introduced thereinto and HA-TCO (2 mg) with the second click chemistry functional group introduced thereinto, which were prepared according to Example 1, were each dissolved in 2% physiological saline to prepare 100 μL of a solution, and 0.2 mg of doxorubicin (Dox) as an anticancer agent was added to each solution. After stirring for 1 hour, the stirred mixed solutions were mixed in a dual syringe to finally obtain a specific cross-linked hyaluronic acid hydrogel (Cx-HA-Dox) containing doxorubicin (0.4 mg).

### Example 6. Preparation of Specific Crosslinked Hyaluronic Acid Hydrogel Containing Therapeutic Agent for Diabetic Disease

HA-Tet (2 mg) with the first click chemistry functional group introduced thereinto and HA-TCO (2 mg) with the second click chemistry functional group introduced thereinto, which were prepared according to Example 1, were each dissolved in 2% physiological saline to prepare 100 μL of a solution, and 31.5 IU of insulin was added to each solution. After stirring for 1 hour, the stirred mixed solutions were mixed in a dual syringe to finally obtain a specific cross-linked hyaluronic acid hydrogel (Insulin-Cx-HA) containing insulin (63 IU).

### Comparative Example 1. Preparation of Hyaluronic Acid Hydrogel

Hyaluronic acid was dissolved in 2% physiological saline, thereby finally preparing a hyaluronic acid hydrogel (HA).

### Comparative Example 2. Preparation of Hyaluronic Acid Hydrogel

with Fluorescent Material Introduced thereinto 3.3 mg of 4-(4,6-dimethoxy-1,3,5-triazin-2-yl)-4-methylmorpholinium chloride (DMTMM) was added as a condensing agent to a vial in which hyaluronic acid was dissolved in 2% physiological saline, and then while being slowly added dropwise to 5 mL of tertiary distilled water containing 10.5 mg of IR783-COOH prepared according to Example 4, the resultant solution was stirred at 25° C. for 24 hours to allow a reaction to occur, followed by dialysis for 3 days and lyophilization at −80° C., thereby completing the preparation of NIR-HA with IR783 introduced thereinto.

### Comparative Example 3. Preparation of Hyaluronic Acid Hydrogel Containing Anticancer Agent

The hyaluronic acid hydrogel (4 mg) prepared according to Comparative Example 1 was dissolved in 2% physiological saline to prepare a 200 solution, and through a syringe, hyaluronic acid hydrogel (HA-Dox) containing doxorubicin (0.4 mg) as an anticancer agent was finally obtained.

### Comparative Example 4. Preparation of Hyaluronic Acid Hydrogel Containing Therapeutic Aunt for Diabetic Disease

The hyaluronic acid hydrogel (4 mg) prepared according to Comparative Example 1 was dissolved in 2% physiological saline to prepare a 200 solution, and through a syringe, hyaluronic acid hydrogel (Insulin-Dox) containing insulin (63 IU) was finally obtained.

### Comparative Example 5. Preparation of Freeze-Dried Pulverized Product of Hyaluronic Acid Hydrogel Crosslinked by Introducing First and Second Click Chemistry Functional Groups

The specific crosslinked hyaluronic acid hydrogel prepared according to Example 1 through a click chemistry reaction was freeze-dried and pulverized in liquid nitrogen using a cryopulverizer (6700, SPEX Inc., USA) under conditions of three cycles of pre-cooling for 50 seconds/grinding (60 Hz) for 240 seconds/cooling for 50 seconds (Pulverized-HA).

### Experimental Example 1. Evaluation of Rheological Properties of Hydrogels

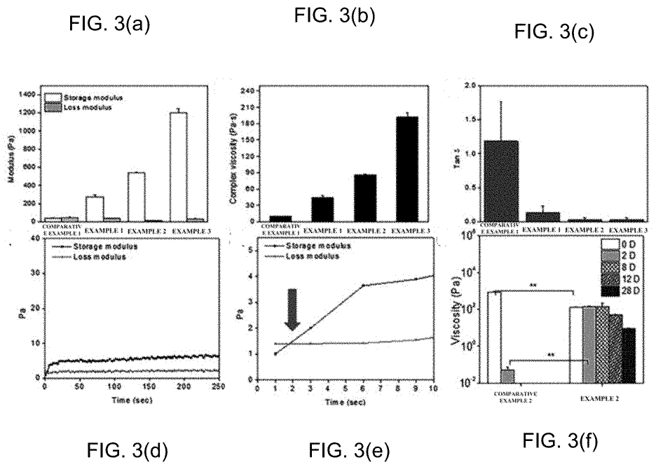

To evaluate the rheological properties of the hyaluronic acid hydrogels prepared through specific crosslinking by a click chemistry reaction in Example 1 and the hyaluronic acid hydrogel prepared according to Comparative Example 1, a modular compact rheometer (MCR 102, Anton Paar, Austria) was used to measure the modulus and viscosity. At this time, the used parallel plate is 25 mm in diameter, the distance from the bottom surface is 0.3 mm, and the measurement was performed under the conditions of a strain of 2% and 1 Hz at 25° C., and the results thereof are shown in FIG. 3.

With regard to the modulus values of the prepared hydrogels, as shown in FIG. 3(a), it was confirmed that the hyaluronic acid hydrogel prepared through specific crosslinking by a click chemistry reaction in Example 1 exhibited a loss modulus almost the same as that of the hyaluronic acid hydrogel prepared according to Comparative Example 1, while exhibiting a significantly increased storage modulus by specific crosslinking through a click chemistry reaction, and it was also confirmed that the intensity of the storage modulus was very significantly increased according to an increase in the degree of click crosslinking.

In addition, in relation to the viscosity of the prepared hydrogels, as shown in FIG. 3(b), it was confirmed that the hyaluronic acid hydrogel prepared according to Example 1 exhibited a much higher viscosity than the hyaluronic acid hydrogel prepared according to Comparative Example 1 did, and it was also confirmed that the viscosity was significantly increased according to an increase in the degree of click crosslinking.

In addition, in relation to the elasticity of the prepared hydrogels measured using a modular compact rheometer (MCR 102, Anton Paar, Austria), as illustrated in FIG. 3(c), it was confirmed that the hyaluronic acid hydrogel prepared according to Example 1 exhibited much higher elasticity than the hyaluronic acid hydrogel prepared according to Comparative Example 1 did, and it was also confirmed that the elasticity was significantly increased according to an increase in the degree of click crosslinking.

As illustrated in FIGS. 3(d) and 3(e), it was observed that the loss modulus and storage modulus of the hydrogel (Example 1, Cx-HA-250) specifically crosslinked through the click chemistry reaction of HA-Tet having the first click chemistry functional group introduced thereinto and HA-TCO having the second click chemistry functional group introduced thereinto were reversed in about 2 seconds. From the results, it can be seen that the specific crosslinking through a click chemistry reaction occurs within 3 seconds.

Meanwhile, it was observed that the loss modulus and storage modulus of Cx-HA-500 (Example 2) and Cx-HA-100 (Example 3) were reversed simultaneously with measurement, and it can be seen that the click reaction was too fast to be measured.

In addition, in the case where each of HA-Tet having the first click chemistry functional group introduced thereinto and NIR-HA-TCO having the second click chemistry functional group introduced thereinto, which were prepared in Examples 1, 2, and 3, was placed in a dual syringe and injected via a syringe, injection was easily performed by pressing the syringe in the case of Example 1, whereas a stronger force was required for injection in the case of Example 3. From the results, it can be seen that, as the amount of introduced Tet or TCO is increased, the degree of crosslinking is increased, and the click reaction proceeds very fast.

### Experimental Example 2. Evaluation of Viscosity Properties of Hydrogels

Each of the hydrogels prepared according to Example 1 (Cx-HA-250) and Comparative Example 2 was placed in a 5 ml vial, and immediately after preparation, and on day 2, day 8, day 12, and day 28 after preparation, the viscosity of each hydrogel was measured using a modular compact rheometer (MCR 102, Anton Paar, Austria).

As a result, as illustrated in FIG. 3(f), it was confirmed that the specific cross-linked hydrogel prepared according to Example 2 through a click chemistry reaction maintained the viscosity for 28 days or longer, whereas the pluronic hydrogel prepared according to Comparative Example 2 maintained the viscosity for 1 day and exhibited very low viscosity from day 2, and after day 3, turned into a solution whose viscosity was unable to be measured.

### Experimental Example 3. Observation of Insides of Hydrogels According to Degree of Crosslinking of Hydrogel

In order to evaluate the degree of internal crosslinking of the hyaluronic acid hydrogels prepared according to Examples 2 and 3 and Comparative Example 1, each hyaluronic acid hydrogel was coated using a plasma-sputtering apparatus (Emitech, K575, Kent, UK), and then the internal structure of each hydrogel was observed using a scanning electron microscope (FE-SEM; JSM-6700F, JEOL, Tokyo, Japan).

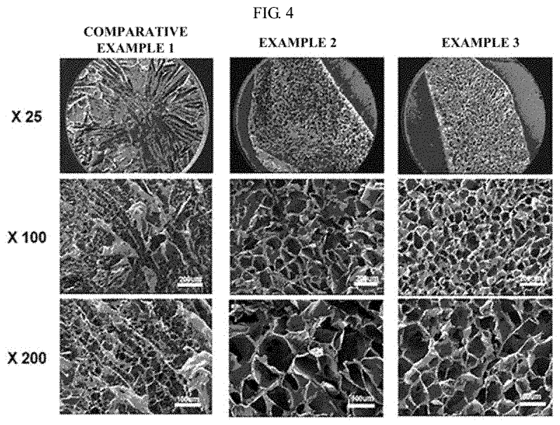

As a result, as illustrated in FIG. 4, it was confirmed that the case of Comparative Example 1 had a structure without porosity due to the agglomeration of internal hydrogel structures, whereas the cases of Examples 2 and 3 maintained a certain porous form. It was also confirmed that the greater the number of first and second click chemistry functional groups (Tet and TCO), the smaller the pore size and the higher the degree of crosslinking between Tet and TCO.

### Experimental Example 4. Observation of Swelling Ratio of Hydrogel to with Respect to Water

In order to evaluate a water swelling property of each of the hyaluronic acid hydrogels prepared according to Examples 1, 2, and 3 and Comparative Example 1 with respect to water, 200 μL of each hyaluronic acid hydrogel was placed in a 20 mL vial and 10 mL of PBS was added thereto, and then the weight of each hydrogel over time was measured to obtain a swelling ratio with respect to water. The swelling ratio with respect to water was calculated using the following equation:

Swelling ratio with respect to water (%)=[(weight of hydrogel absorbing water-weight of dried hydrogel)/(weight of dried hydrogel)]×100

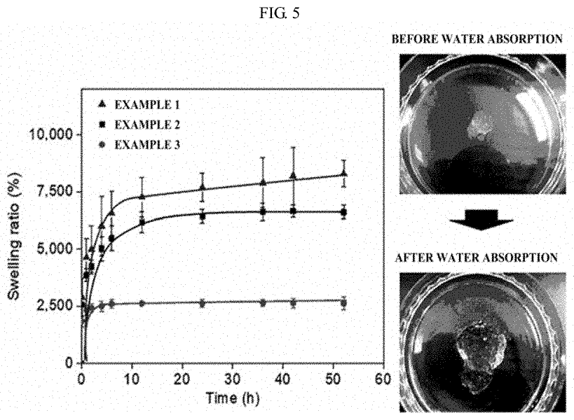

As a result, as illustrated in FIG. 5, it was confirmed that the hyaluronic acid hydrogel of Comparative Example 1 became a solution within a few minutes after adding PBS, so that the swelling ratio with respect to water could not be calculated, whereas the hyaluronic acid hydrogel prepared in Example 1 exhibited the greatest swelling ratio with respect to water, and the swelling ratio with respect to water was significantly reduced in accordance with an increase in the degree of click crosslinking.

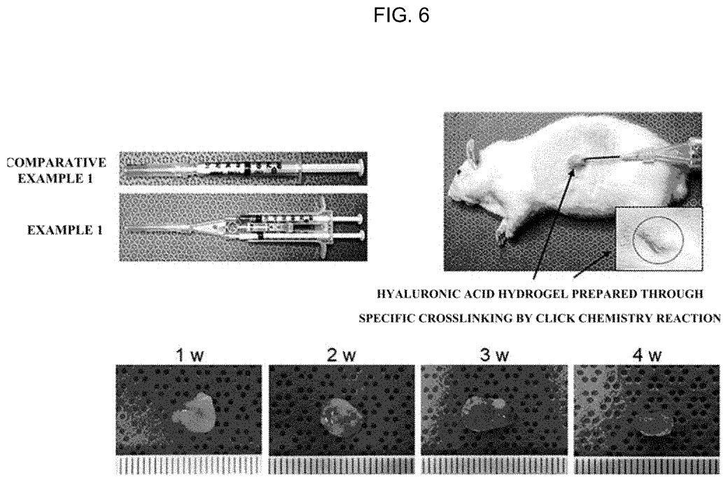

Experimental Example 5. Observation of Hydrogel Formation Each of the prepared HA-Tet and HA-TCO was added to a dual syringe, and 100 μl of each hydrogel was injected subcutaneously into mice to form a hydrogel under the skin. Thereafter, the hydrogel was extracted from under the skin of each mouse at intervals of 1 week and observed. As a result, as illustrated in FIG. 6, it can be seen that, even after 4 weeks, the gel was stably formed.

Meanwhile, 100 μl of the hyaluronic acid hydrogel prepared in Comparative Example 1 was subcutaneously injected into each mouse to form a hydrogel and observed, but gel formation could not be confirmed from 1 week on.

### Experimental Example 6. Observation of Hydrogel Formation Through Near-Infrared Fluorescence Imaging

NIR-HA-Tet having the first click chemistry functional group and IR783 introduced thereinto and NIR-HA-TCO having the second click chemistry functional group and IR783 introduced thereinto, which were prepared according to Example 4, were each put in a dual syringe and 0.15 mL thereof was subcutaneously injected into each nude mouse (male, 8-week-old) via a 1 mL syringe (21 G). For a comparative experiment, NIR-HA prepared according to Comparative Example 3 was injected into each nude mouse using the same method as described above, and then fluorescence imaging over time was observed.

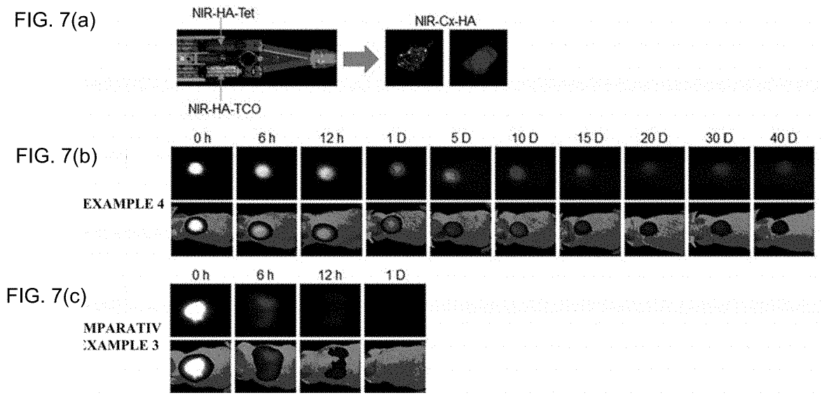

As a result, as illustrated in FIG. 7, it can be seen that, while the fluorescence imaging of the hyaluronic acid hydrogel prepared according to Example 4 was maintained for 40 days or longer (FIG. 7B), the fluorescence imaging of the injected hyaluronic acid hydrogel prepared according to Comparative Example 3 was maintained up to 12 hours, and was not observed any longer after 1 day (FIG. 7C).

### Experimental Example 7. Drug Release Effect of Hydrogel Containing Anticancer Agent

The anticancer agent-containing hydrogels prepared according to Example 5 and Comparative Example 3 were each dissolved in 0.5 mL of PBS, and then injected into the upper chamber of a 24-well Transwell plate (SPL Life Sciences, 0.4 μm pore size) to confirm the release of the drug over time in a shaker at 37° C. and 100 rpm. 0.5 mL of the solution was collected according to each time point, and then 0.5 mL of a PBS solution was injected again. The release of doxorubicin was measured using a high performance liquid chromatography (HPLC) system (Agilent 1200 series, Waldbronn, Germany). To measure the amount of doxorubicin, a wavelength of 479 nm and a CAPCELL PACK C18 column (5 μm, 4.6×250 mm, Shiseido Co., Ltd., Tokyo, Japan) were used. A mixed solution of 50 mM sodium phosphate and acetonitrile at a volume ratio of 30:70 (v/v) was used as a mobile phase, and a flow rate was 0.5 mL/min. The cumulative doxorubicin release rate in vitro was calculated by comparison with a standard calibration curve.

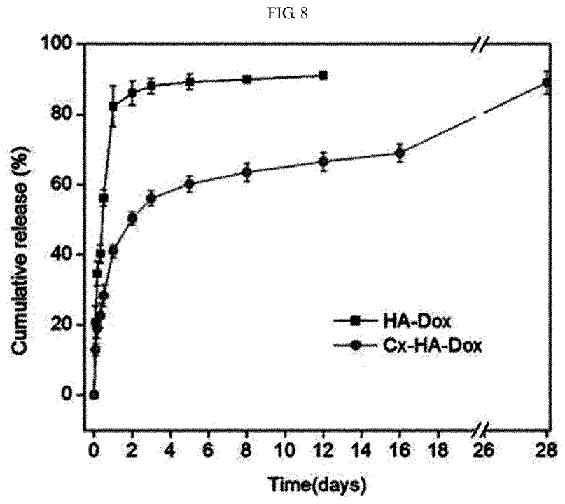

As a result, as illustrated in FIG. 8, it was confirmed that the anticancer agent-containing specific crosslinked hydrogel (Cx-HA-Dox) prepared according to Example 5 exhibited a sustained release of doxorubicin, as compared to the anticancer agent-containing hydrogel prepared according to Comparative Example 3, and a cumulative doxorubicin release rate for 16 days was confirmed to be about 60% to about 70%.

After the release test for 28 days, as a result of measuring the content of doxorubicin remaining in the anticancer agent-containing specific crosslinked hydrogel (Cx-HA-Dox) prepared according to Example 5, the content was confirmed to be about 10.4%. Accordingly, it is determined that the actual cumulative doxorubicin release rate is about 90%.

### Experimental Example 8. Cellular Anticancer Effect of Hydrogel Containing Anticancer Agent

2×10⁴ B16F10 melanoma cells were seeded in each lower chamber of a 24-well Transwell plate (SPL Life Sciences, 0.4 μm pore size) and incubated for one day at 37° C. in 5% CO₂. Then, 200 μL of PBS, doxorubicin (Dox; 0.4 mg Dox/200 μL), doxorubicin repeat (0.1 mg Dox/200 μL), the hydrogel prepared according to Comparative Example 1 (HA; 4 mg HA/200 μL), the anticancer agent-containing hydrogel prepared according to Comparative Example 3 (HA-Dox; 4 mg HA, 0.4 mg Dox/200 μL), the specific crosslinked hydrogel prepared according to Example 1 (Cx-HA; 2 mg TET-HA, 2 mg TCO-HA/200 μL), and the anticancer agent-containing specific crosslinked hydrogel prepared according to Example 5 (Cx-HA-Dox; 2 mg TET-HA, 2 mg TCO-HA, 0.4 mg Dox/200 μL) were added to each upper chamber of the 24-well Transwell plate. Each well containing the cells was covered with the upper chamber, and the cells were incubated at 37° C. in 5% CO₂ for 12 hours, 24 hours, 48 hours, and 72 hours, and after 12 hours, the initially added media were replaced. In order to confirm the cytotoxicity of each formulation against the B16F10 cells in vitro for 12 hours, 24 hours, 48 hours, and 72 hours, MTT (3-(4,5-dimethylthiazol-2-yl)-2,5-diphenyltetrazolium bromide; Sigma-Aldrich Co., St. Louis, Mo., USA) assay was performed. 100 μL of a PBS solution containing an MTT tetrazolium substrate (50 μg/mL) was added to each well, followed by incubation at 37° C. for 4 hours. At this time, in order to dissolve the produced purple formazan, 500 μL/well of DMSO was injected and shaken for 30 minutes, and then the solution was transferred to a 96-well plate. The optical density of the solution at a wavelength of 590 nm was measured using a microplate reader (E-max, Molecular Devices, Sunnyvale, Calif., USA).

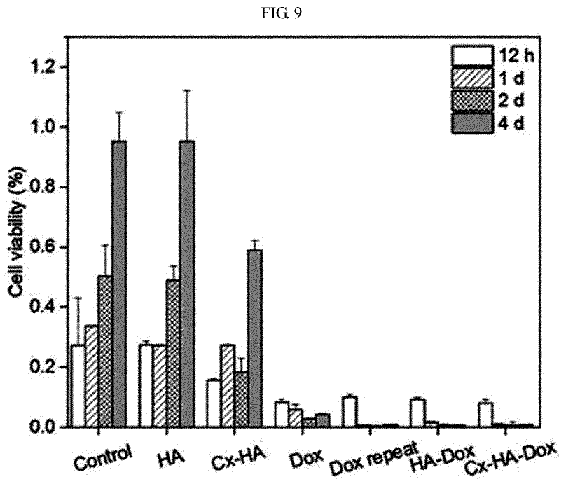

As a result, as illustrated in FIG. 9, it is confirmed that, when the anticancer agent is contained, the cellular anticancer effect is excellent. However, it is confirmed that, even when the anticancer agent is not contained, the specific crosslinked hydrogel (Cx-HA) prepared according to Example 1 exhibited an enhanced cellular anticancer effect, as compared to the hydrogel (HA) prepared according to Comparative Example 1.

### Experimental Example 9. Comparison of Cancer Sizes of Anticancer Agent-Containing Hydrogels when Applied to Animals

A cancer model was produced by subcutaneously injecting 100 μL of a suspension of about 2×10⁵ B16F10 melanoma cells into the abdomen of BALB/c mice (6-week-old, male, 20 g). The case where the tumor volume became 150-200 mm³ was assumed to be 0 d, and cancer-induced animals were divided into six experimental groups: (1) Control; (2) doxorubicin (Dox; 0.4 mg Dox); (3) Dox repeat (0.1 mg Dox); (4) the anticancer agent-containing hydrogel prepared according to Comparative Example 3 (HA-Dox; 0.4 mg Dox); (5) the anticancer agent-containing specific crosslinked hydrogel prepared according to Example 5 (Cx-HA-Dox; 0.4 mg Dox); and (6) a composition containing an anticancer agent (doxorubicin) in the freeze-dried pulverized product of the specific crosslinked hydrogel prepared according to Comparative Example 5 (Pulverized-HA; 0.4 mg Dox). When the tumor volume reached 150-200 mm³, each solution was injected into the tumor using a 1 mL syringe and a 21-gauge needle. At this time, the injection rate of the solution was maintained at 10 μL/s to prevent the solution from flowing into the surrounding tissues. Anti-tumor activity was evaluated by two-dimensionally measuring the tumor diameter using Vernier calipers on a predefined date. The tumor volume (V) was calculated using the following equation: V=[length×(width)²]/2.

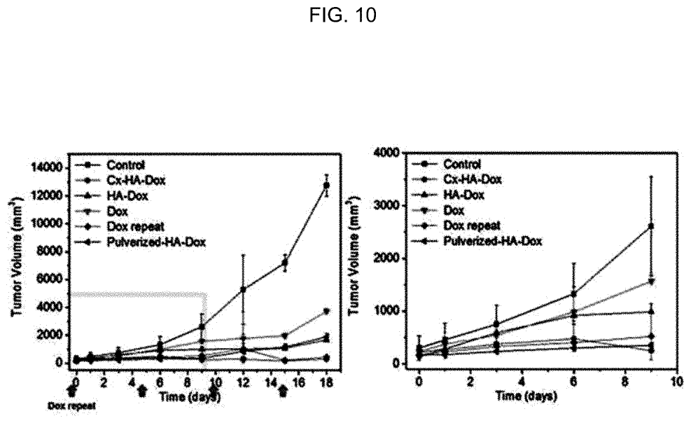

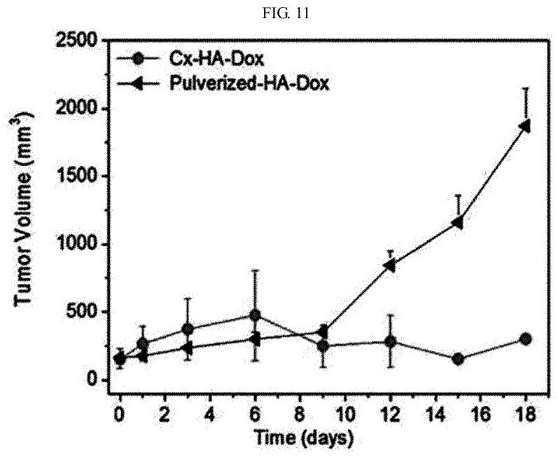

As a result, as illustrated in FIGS. 10 and 11, it is confirmed that the anti-tumor activity was remarkably improved when the anticancer agent is contained. In particular, it is confirmed that the anticancer agent-containing specific crosslinked hydrogel (Cx-HA-Dox) prepared according to Example 5 exhibits further enhanced antitumor activity, as compared to the anticancer agent-containing hydrogel (HA-Dox) prepared according to Comparative Example 3 and the anticancer agent-containing hydrogel prepared according to Comparative Example 5 (Pulverized-HA-Dox), and thus the hydrogel of Example 5 is expected to be applicable as an effective drug carrier.

### Experimental Example 10. Anticancer Pharmacokinetic Effect of Anticancer Agent-Containing Hydrogel when Applied to Animals

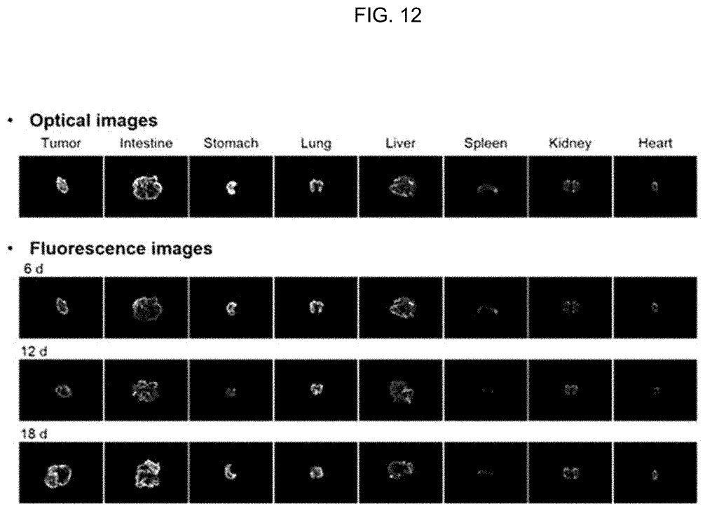

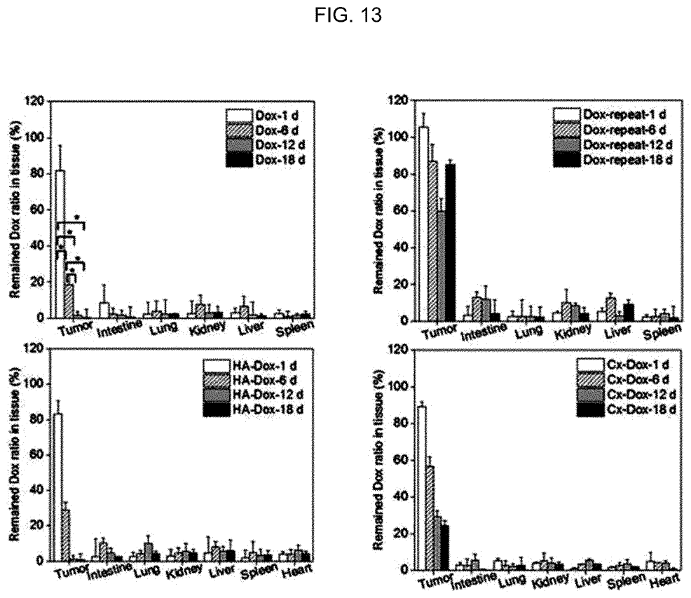

To perform a pharmacokinetic experiment, mice were sacrificed on day 1, day 6, day 12, and day 18. Organs (tumors, the intestines, lungs, kidneys, liver, spleen, and heart) were immediately acquired and photographed (FIG. 12). To measure the amount of doxorubicin remaining in each organ, each organ was homogenized in a 0.3N HCl/70% ethanol solution at 25,000 rpm to 30,000 rpm using a T 10 basic ULTRA-TURRAX Homogenizer (IKA-Werke GmbH & Co., Staufen, Germany), followed by incubation at 37° C. for 15 minutes. Each uniformly mixed sample was mixed with the same volume of 40% ZnSO₄ and a mobile phase, and incubated again at 37° C. for 15 minutes. When the incubation was completed, each sample was centrifuged at 2,000 rpm for 10 minutes, and the amount of doxorubicin contained in the supernatant was calculated by referring to the standard calibration curve. The amount of doxorubicin contained in each organ was measured by a UV-visible Spectrophotometer (V-770, JASCO Inc., Tokyo, Japan) (FIG. 13).

As a result, as illustrated in FIG. 13, it is confirmed that, in the case of injection of doxorubicin (Dox) alone, there is a problem in that the anticancer agent is distributed more rapidly to various organs than a tumor. Meanwhile, it is confirmed that, in the case of Dox repeat injection, a large amount of anticancer agent is present in a tumor and, compared to the injection of doxorubicin alone, a large amount of anticancer agent remains in various organs and affects the same, and thus the repeated injection of an anticancer agent results in anticancer treatment, but also induces various side effects due to the anticancer agent.

It is also confirmed that, in the case of injection of the anticancer agent-containing hydrogel (HA-Dox) prepared according to Comparative Example 3, the anticancer agent is present only at the early time of a tumor and rapidly distributed to other organs, and thus there is no efficacy as a drug carrier.

In contrast, it is confirmed that, in the case of injection of the anticancer agent-containing specific crosslinked hydrogel (Cx-HA-Dox) prepared according to Example 5, the amount and residence time of the anticancer agent present in a tumor are greater than those of the other experimental groups, and the occurrence of various side effects on various organs due to the anticancer agent is prevented or reduced, and thus the hydrogel of Example 5 is expected to be applicable as an effective drug carrier.

### Experimental Example 11. Preparation of Hydrogel Containing Therapeutic Agent for Diabetic Disease

A diabetic disease model was produced by dissolving streptozotocin (40 mg/kg) in citrate buffer (pH 4.5) and injecting the resultant buffer via the tail, and on day 2 after injection, a blood glucose level of 380 to 420 was confirmed. The hydrogels prepared according to Example 6 and Comparative Example 4 containing a therapeutic agent for diabetic disease were administered via subcutaneous injection, and blood was collected for 18 days in the morning (around 10 a.m.), and the blood glucose level was measured using an Accu-Chek Advantage meter.

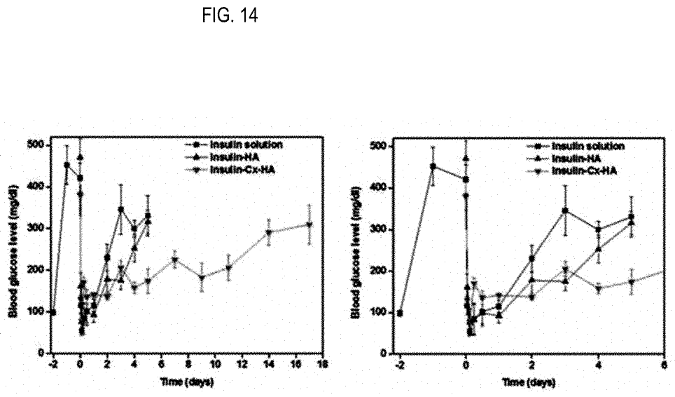

As a result, as illustrated in FIG. 14, it is confirmed that, in the case of the specific crosslinked hydrogel (Insulin-Cx-HA) containing the therapeutic agent for diabetic disease prepared according to Example 6, the blood glucose level is stably maintained as compared to the insulin-containing hydrogel (Insulin-HA) prepared according to Comparative Example 4, and thus the hydrogel of Example 6 is expected to be applicable as an effective drug carrier.

1. An injectable composition comprising:
a first liquid comprising a first biopolymer into which a first click chemistry functional group is introduced; and
a second liquid comprising a second biopolymer into which a second click chemistry functional group is introduced,
wherein the first click chemistry functional group and the second click chemistry functional group are chemically linkable.

2. The injectable composition of claim 1, wherein the first click chemistry functional group comprises one or more selected from an alkyne group, an epoxy group, an acryloyl group, and a tetrazine group, and the second click chemistry functional group comprises one or more selected from an azide group, a thiol group, an amine group, and a cyclooctene group.

3. The injectable composition of claim 1, wherein a combination of the first click chemistry functional group and the second click chemistry functional group comprises one or more selected from: an alkyne group and an azide group; an alkyne group and a thiol group; an epoxy group and an amine group; an epoxy group and a thiol group; an acryloyl group and an amine group; an acryloyl group and a thiol group; and tetrazine and cyclooctene.

4. The injectable composition of claim 1, wherein the first biopolymer and the second biopolymer are the same or different from each other, and each independently comprises one or more selected from hyaluronic acid, small intestine submucosal tissue, carboxymethylcellulose, alginate, chitosan, a pluronic, polyacrylamide, poly(N-isopropylacrylamide), and β-glycerophosphate.

5. The injectable composition of claim 1, wherein a drug is additionally included in the first liquid and the second liquid.

6. The injectable composition of claim 5, wherein the drug comprises an anticancer agent comprising one or more selected from doxorubicin, cisplatin, paclitaxel, vincristine, topotecan, docetaxel, 5-fluorouracil (5-FU), Gleevec, carboplatin, daunorubicin, valrubicin, flutamide, and gemcitabine; or a therapeutic agent for a diabetic disease comprising insulin or an insulinotropic peptide.

7. The injectable composition of claim 1, wherein the number of first click chemistry functional groups introduced with respect to 1 mole of the first biopolymer or the number of second click chemistry functional groups introduced with respect to 1 mole of the second biopolymer is in a range of 100 to 2,000.

8. A method of preparing an injectable hydrogel, the method comprising:
(a) preparing a first liquid by adding a material comprising a first click chemistry functional group to a first biopolymer;
(b) preparing a second liquid by adding a material comprising a second click chemistry functional group to a second biopolymer; and
(c) reacting the first liquid and the second liquid to chemically link the first click chemistry functional group and the second click chemistry functional group.

9. The method of claim 8, wherein, in process (a), the material comprising a first click chemistry functional group comprises one or more selected from amino-PEG4-alkyne, alkyne-PEG5-acid, alkyne-PEG-amine, oxiranylamine, 2-oxiranyl-ethylamine, acrylamide, acrylic acid, acryloyl chloride, methyltetrazine-amine, methyltetrazine-PEG4-amine, methyltetrazine-propylamine, tetrazine-PEG5-NHS ester, methyltetrazine-PEG4-NHS ester, methyltetrazine-silfo-NHS ester, methyltetrazine-PEG4-acid, methyltetrazine-PEG12-NHS ester, methyltetrazine-NHS ester, methyltetrazine-acid, and tetrazine-acid, and in process (b), the material comprising a second click chemistry functional group comprises one or more selected from azide-PEG4-amine, 3-amino-1-propanethiol, 11-mercaptoundecanoic acid, amino-methanethiol, thiol PEG amine, ethylene diamine, PEG diamine, (S)-3-amino-2-(hydroxymethyl)propionic acid, amino-acetic acid, transcyclooctene-amine, trans-cyclooctene-NHS ester, trans cyclooctene-PEG-NHS ester, and trans cyclooctene-PEG4-acid.

10. The method of claim 8, wherein, in process (a) or (b), a condensing agent or a drug is further added.

11. The method of claim 8, wherein the injectable hydrogel has a storage modulus (G′) of 2.00×10² Pa to 2.00×10³ Pa in a frequency range of 0.1 Hz to 10 Hz, the storage modulus (G′) being measured using a rheometer.

12. The method of claim 8, wherein the injectable hydrogel has a complex viscosity of 3.00×10¹ Pas to 3.0×10² Pas at 25° C.

13. The method of claim 8, wherein the injectable hydrogel is in a porous form.

14. The method of claim 8, wherein a swelling ratio of the injectable hydrogel with respect to water is in a range of 2,000% to 10,000%.

15. A medical filler or bio-injectable scaffold comprising an injectable hydrogel prepared according to the method of claim 8.

16. A drug carrier comprising an injectable hydrogel prepared according to the method of claim 8 and a drug.

17. The drug carrier of claim 16, wherein the drug comprises an anticancer agent comprising one or more selected from doxorubicin, cisplatin, paclitaxel, vincristine, topotecan, docetaxel, 5-fluorouracil (5-FU), Gleevec, carboplatin, daunorubicin, valrubicin, flutamide, and gemcitabine; or a therapeutic agent for a diabetic disease comprising insulin or an insulinotropic peptide.

18. The drug carrier of claim 16, wherein the drug carrier has a cumulative drug release rate of 50% to 100% for 28 days.
